(19) United States
(12) Patent Application Publication
(10) Pub. No.: US 2023/0125093 A1
(43) Pub. Date: Apr. 27, 2023

(54) METHODS FOR ANION-EXCHANGE ANALYSIS OF NUCLEIC ACIDS

(71) Applicant: Waters Technologies Corporation, Milford, MA (US)

(72) Inventors: Matthew A. Lauber, North Smithfield, RI (US); Kevin Wyndham, Upton, MA (US); Szabolcs Fekete, Ville La Grand (FR); Hua Yang, Hopkinton, MA (US); Stephan Koza, Lancaster, MA (US)

(73) Assignee: Waters Technologies Corporation, Milford, MA (US)

(21) Appl. No.: 17/970,874

(22) Filed: Oct. 21, 2022

Related U.S. Application Data
(60) Provisional application No. 63/275,496, filed on Nov. 4, 2021.
(60) Provisional application No. 63/270,098, filed on Oct. 21, 2021.

(51) Int. Cl. C12N 15/10

(52) U.S. Cl. CPC C12N 15/101

(57) ABSTRACT

The present invention relates to a method for separating a sample comprising a nucleic acid, the method comprising: (a) loading a sample comprising a nucleic acid onto an anion-exchange column; and (b) eluting the sample from the anion-exchange column using a salt gradient, wherein a mobile phase used in the eluting step comprises a mild ion-pairing cation, and wherein a column temperature of the anion-exchange column is greater than 30° C. The present invention also relates to a method for determining one or more calorimetric properties of a nucleic acid using anion-exchange chromatography.

### CROSS-REFERENCE TO RELATED APPLICATION

This application claims priority and benefit to U.S Provisional Pat. Applications No. 63/270,098, filed Oct. 21, 2021 and 63/275,496, filed Nov. 4, 2021, both of which are entitled “METHODS FOR ANION-EXCHANGE ANALYSIS OF NUCLEIC ACIDS”. The contents of each are incorporated herein by reference in their entirety.

### FIELD OF THE INVENTION

The present disclosure relates to a method for the high resolution separation of a sample comprising a nucleic acid using anion-exchange chromatography. The present disclosure also relates to a method for determining one or more calorimetric properties of a nucleic acid using anion-exchange chromatography.

### BACKGROUND OF THE INVENTION

mRNA, a transcript form of DNA read by ribosomes within cells during the process of translation, i.e. protein production, represents a new class of advanced medicinal products. For example, mRNA vaccines have recently been used to immunise millions of people across the world against SARS-CoV-2. As medicine advances further and mRNA and other nucleic acids are increasingly used in medicinal products, e.g. as drug candidates, a need for sensitive and informative methods for characterizing the biophysical properties of nucleic acids arises.

Due to the repeated phosphate groups in the sugar-phosphate backbone of nucleic acids, nucleic acids are negatively charged. Anion-exchange chromatography is a chromatographic technique used for separating negatively charged analytes. To date, very few analytical scale anion-exchange separations of nucleic acids, particularly RNA, have been published. Kanavarioti, Scientific Reports 1019 (2019) discloses some of the few examples of analytical scale anion-exchange separations of RNA. Kanavarioti discloses anion-exchange separations using a pellicular particle stationary phase and either a column temperature of 10° C. and a pH of 12, or a column temperature of 60° C. and a neutral pH. The separation conditions used in Kanavarioti, i.e. relatively high temperature or very high pH, are unlikely to be ideal for separating nucleic acid containing samples while also preserving the nucleic acid such that the heterogeneity of the intact nucleic acid can be optimally profiled. Accordingly, there exists the need for an improved method for separating a sample comprising a nucleic acid. The inventors of the present disclosure have developed two such methods, which are disclosed herein.

The inventors of the present disclosure have also developed a method for determining one or more calorimetric properties of a nucleic acid using anion-exchange chromatography. Said method provides an alternative to differential scanning calorimetry (DSC) for determining calorimetric properties of nucleic acids.

### SUMMARY OF THE INVENTION

According to the present invention there is provided a method for separating a sample comprising a nucleic acid, the method comprising:
(a) loading a sample comprising a nucleic acid onto an anion-exchange column; and
(b) eluting the sample from the anion-exchange column using a salt gradient,
   wherein a mobile phase used in the eluting step comprises a mild ion-pairing cation, and
   wherein a column temperature of the anion-exchange column is greater than 30° C.

Preferably, the salt gradient is produced by a salt comprising the mild ion-pairing cation.

Advantageously, the salt gradient is produced by a non-ion-pairing salt, preferably a chloride or a bromide containing salt, wherein a concentration of the mild ion-pairing cation remains constant throughout the eluting step. Preferably, the concentration of the mild ion-pairing cation is 10 to 2,000, 10 to 1,000 or 10 to 500 mM.

When the salt gradient is produced by a non-ion-pairing salt and the concentration of the mild ion-pairing cation remains constant throughout the eluting step it is preferable that the column temperature does not exceed 50° C. and more preferable that it does not exceed 45° C.

Conveniently, the salt gradient is produced by: a salt comprising the mild ion-pairing cation; and a non-ion-pairing salt, preferably a chloride or a bromide containing salt.

Preferably, the nucleic acid is RNA. More preferably, the nucleic acid is mRNA.

Advantageously, the method uses at least two mobile phases, and optionally three mobile phases, wherein the mobile phases are buffered with a basic or a zwitterionic buffering agent.

Conveniently, the buffering agent is selected from Tris, Bis-tris propane, MES, and HEPES, though other basic or zwitterionic buffering agents may equally be employed.

Preferably, the buffering agent is Tris.

Advantageously, the mild ion-pairing cation is nitrogenous.

Conveniently, the mild ion-pairing cation is tetramethylammonium. In preferred aspects of the invention, the salt comprising the mild ion-pairing cation is tetramethylammonium chloride. In some aspects of the invention, the salt comprising the mild ion-pairing cation is an alternative tetramethylammonium cation-containing salt.

Preferably, the column temperature of the anion-exchange column is from 40° C. to 60° C. In aspects of the invention, wherein the salt used in the salt gradient is tetramethylammonium chloride, the column temperature is preferably 40° C.

Advantageously, the method uses at least two mobile phases, and optionally three mobile phases, wherein the mobile phases have a pH of from 6.5 to 10. Preferably, the mobile phases have a pH of from 7 to 9.

Conveniently, the method uses at least two mobile phases, and optionally three mobile phases, wherein one or more of the at least two mobile phases comprises at least one additional mild ion-pairing cation. Preferably, the at least one additional mild ion-pairing cation is selected from tetramethylammonium, triethylammonium, diisopropylethylammonium, or combinations thereof.

According to another aspect of the present invention there is provided a method for determining one or more calorimetric properties of a nucleic acid, the method comprising:
a) loading a sample comprising a nucleic acid onto an anion-exchange column;
b) eluting the sample from the anion-exchange column at a first column temperature to produce a first chromatogram;
c) repeating steps (a) and (b) for a plurality of additional column temperatures to produce a corresponding plurality of additional chromatograms; and
d) comparing the chromatograms obtained at different column temperatures to determine one or more calorimetric properties of the nucleic acid.

Preferably, step (c) comprises repeating steps (a) and (b) for at least 3 additional column temperatures.

Advantageously, step (d) comprises comparing a peak area of a peak produced by the nucleic acid in the first chromatogram to a peak area of a corresponding peak in each of the plurality of additional chromatograms.

Conveniently, step (d) further comprises plotting a graph of peak area or relative peak area against column temperature.

Preferably, between step (b) and step (c), the method further comprises the step (e) of performing a blank run at the first column temperature to produce a first blank run chromatogram; and step (c) comprises repeating steps (a), (b) and (e) for a plurality of additional column temperatures to produce a corresponding plurality of additional chromatograms and additional blank run chromatograms.

Advantageously, step (d) comprises comparing the chromatogram and blank run chromatogram produced at each column temperature to determine a carry-over % of the nucleic acid for each column temperature, and comparing the carry-over % for each column temperature.

Conveniently, step (d) further comprises plotting a graph of carry-over % against column temperature.

### BRIEF DESCRIPTION OF THE DRAWINGS

FIG. 1A shows an illustrative plot of relative recovery % against temperature obtained by carrying out an anion-exchange separation of EPO mRNA at a variety of different temperatures between 30° C. and 70° C.

FIG. 1B shows an illustrative plot of carry-over % against temperature obtained by carrying out an anion-exchange separation of EPO mRNA at a variety of different temperatures between 30° C. and 70° C.

FIG. 2A, FIG. 2B, FIG. 2C and FIG. 2D are chromatograms obtained for the anion-exchange separation of Cas9 mRNA, using a NaCl salt gradient, at four different column temperatures between 30° C. and 60° C.

FIG. 2E, FIG. 2F, FIG. 2G and FIG. 2H are chromatograms obtained for the anion-exchange separation of EPO mRNA, using a NaCl salt gradient, at four different column temperatures between 30° C. and 60° C.

FIG. 3A, FIG. 3B, FIG. 3C and FIG. 3D are chromatograms obtained for the anion-exchange separation of Cas9 mRNA, using a tetramethylammonium chloride salt gradient, at four different column temperatures between 30° C. and 60° C.

FIG. 3E, FIG. 3F, FIG. 3G and FIG. 3H are chromatograms obtained for the anion-exchange separation of EPO mRNA, using a tetramethylammonium chloride salt gradient, at four different column temperatures between 30° C. and 60° C.

FIG. 4 shows chromatograms obtained for the anion-exchange separations of a low range RNA ladder, HPRT sgRNA, GUAC ssRNA, Rosa26 sgRNA, and Scramble #2 sgRNA, using a tetramethylammonium chloride salt gradient, at a column temperature of 60° C.

FIG. 5 shows an illustrative plot of relative recovery % against temperature obtained by carrying out an anion-exchange separation of EPO mRNA and Cas9 mRNA at a variety of different temperatures between 30° C. and 70° C.

FIG. 6A and FIG. 6B are chromatograms obtained for the anion-exchange separation of Cas9 mRNA and EPO mRNA, using a NaCl salt gradient, at 40° C. in the presence of TMAC at static concentration in the mobile phase.

### DETAILED DESCRIPTION OF THE INVENTION

### Method for Separating a Sample Comprising a Nucleic Acid

The present invention provides a method for separating a sample comprising a nucleic acid using anion-exchange chromatography. Herein, “nucleic acid” is used to refer to larger DNA and RNA molecules as well as shorter oligonucleotides.

The sample may comprise more than one nucleic acid.

In preferred aspects of the invention, the method is capable of the high resolution separation of a nucleic acid under conditions that preserve the nucleic acid such that the heterogeneity of the intact nucleic acid can be optimally profiled.

The method comprises the step of loading a sample comprising a nucleic acid onto an anion-exchange column. Typically, the sample is loaded by injection onto the column through an injection valve. The sample may require clean-up, filtration, concentration, or other pre-analysis preparation prior to loading onto the anion-exchange column.

Anion-exchange columns suitable for performing separations according to the invention are known in the art and can be selected without undue experimentation. For example, a non-porous quaternary amine anion-exchange column, such as a Protein-Pak Hi-Res Q, 100 × 4.6 mm, 5 µm column (Waters Corporation, Milford, MA, USA), can be used. Other known anion-exchange columns are also suitable for performing separations according to the invention.

The size of the column can be selected according to factors such as the amount of sample to be analysed. For example, for analysis of very small amounts of sample, a microbore column, a capillary column, or a nanocolumn may be used.

Once the sample has been successfully loaded onto the anion-exchange column, the sample is eluted from the column using a salt gradient. Importantly, in the method of separation according to the present invention, a mobile phase used in the elution step comprises a mild ion-pairing cation. The mild ion-pairing cation is able to exert an ion-pairing effect on the nucleic acid, thereby decreasing the strength of the interaction between the negatively charged nucleic acid and the positively charged anion-exchange column.

Herein, ‘mild ion-pairing cation’ is used to refer to a cation comprising from 3 to 16 carbon atoms, preferably from 3 to 10 carbon atoms, more preferably from 3 to 8 carbon atoms, and most preferably from 3 to 6 carbon atoms.

Preferred mild ion-pairing cations for carrying out the method of the present invention are nitrogenous cations, i.e., cations comprising a positively charged nitrogen atom.

An especially preferred mild ion-pairing cation for use in the method of the present invention is tetramethylammonium. Triethylammonium and diisopropylammonium are examples of other preferred mild ion-pairing cations. However, mild ion-pairing cations that may be used in the method of separation according to the present invention are not limited to substituted amine cations.

In a preferred aspect of the invention, the salt used to perform the salt gradient comprises the mild ion-pairing cation. For example, in preferred aspects of the invention, the salt used to perform the salt gradient is tetramethylammonium chloride or another salt comprising the tetramethylammonium cation.

In another aspect of the invention, the salt used to perform the salt gradient is a non-ion-pairing salt. Herein “non-ion-pairing salt” is used to refer to a salt that does not comprise a mild ion-pairing cation and is not able to exert an ion-pairing effect on charged analytes, such as nucleic acid, in the sample. In such aspects of the invention, the concentration of the mild ion-pairing cation on the anion-exchange column may be kept constant throughout the elution step. For example, the concentration of the mild ion-pairing cation may be maintained at 10 to 500 mM throughout the elution step.

Aspects of the invention where a non-ion-pairing salt is used to perform the salt gradient and where a mild ion-pairing cation is also present in the mobile phase are referred to herein as “ion-pair mediated salt gradient” methods. In such methods the mild ion-pairing cation is present as a mobile phase additive to partially form ion-pairs with the nucleic acid molecules thus decreasing the number of accessible charges of the molecules.

In a further aspect of the invention, the salt gradient may be performed using both a salt comprising the mild ion-pairing cation and a non-ion-pairing salt, i.e. a dual gradient may be used. The concentration of both the salt comprising the mild ion-pairing cation and the non-ion-pairing salt is increased during the elution step.

Preferred non-ion-pairing salts for use in a separation method according to the present invention include chloride and bromide salts such as NaCl and NaBr.

The salt(s) selected for performing the salt gradient may be chosen to optimize the separation of the nucleic acid in the sample.

The salt gradient used to elute the sample need not be linear. For example, nonlinear, multi-linear, and multi-isocratic gradients may also be used. Where both a non-ion-pairing salt and a salt comprising a mild ion-pairing cation are used to perform the salt gradient, the gradient used for each salt need not be the same. The gradient selected for the elution can be optimised to achieve the highest resolution for a particular sample. Empirical, mechanistic retention models may be developed, based on initial separation experiments for a particular sample, allowing the gradient shape to be optimized in silico for said sample.

Typically, the salt(s) for performing the salt gradient is present in just one of the mobile phases used to perform the separation. The salt gradient is produced by varying the percentage of total mobile phase made up of the mobile phase comprising the salt(s) for performing the salt gradient.

In preferred aspects of the invention, the method is carried out using just two mobile phases: a first mobile phase not comprising the salt(s) for performing the salt gradient; and a second mobile phase comprising the salt(s) for performing the salt gradient. To create the salt gradient, the ratio of second mobile phase: first mobile phase run through the anion-exchange column is increased during the elution step.

Alternatively, when performing ion-pair mediated salt gradient methods of the invention as defined above, the mild ion-pairing cation may be provided in a third mobile phase in addition to a first mobile phase not comprising the salt(s) for performing the salt gradient; and a second mobile phase comprising the salt(s) for performing the salt gradient.

The mobile phases used in the separation can be selected to optimize the separation of the nucleic acid in the sample.

In preferred aspects of the invention, one or more of the mobile phases used in the separation may comprise one or more additional mild ion-pairing cations. For example, one or more of the mobile phases may comprise one or more additional mild ion-pairing cations selected from tetramethylammonium, triethylammonium, diisopropylethylammonium, or combinations thereof. In other words, more than one mild ion-pairing cation may be present on the anion-exchange column during the elution step.

To optimize the separation of the nucleic acid in the sample, a mobile phase comprising salt(s) for performing the salt gradient may comprise varying amounts of one or more additional mild ion-pairing cations, these additional mild ion-pairing cations also exerting an ion-pairing effect on the nucleic acid in the sample. In some aspects of the invention, one or more of the mobile phases not comprising salt(s) for performing the salt gradient may additionally comprise varying amounts of one or more additional mild ion-pairing cations.

Preferably, the mobile phases used in the method of separation according to the invention are buffered with a buffering agent that is either basic or zwitterionic, rather than acidic. Such buffering agents do not contribute to the elution strength of the mobile phases used in the separation. Additionally, zwitterionic buffering agents help to control the pH of the positively charged anion-exchange column surface, preventing local pH gradients from developing.

In preferred aspects of the invention, the mobile phases are buffered with a buffering agent selected from Tris (tris(hydroxymethyl)aminomethane), Bis-tris propane (1,3-bis(tris(hydroxymethyl)methylamino)propane), (MES (2-(N-morpholino)ethanesulfonic acid), and HEPES (4-(2-hydroxyethyl)-1-piperazineethanesulfonic acid). In especially preferred aspects of the invention, the mobile phases are buffered with Tris.

Preferably, the method of separation of the invention is carried out at a pH from 6.5 to 10. More preferably, the separation of the invention is carried out at a pH from 7 to 9. A more moderate pH, i.e., a pH from 6.5 to 10 and preferably from 7 to 9, helps preserve the nucleic acid such that the heterogeneity of the intact nucleic acid can be optimally profiled. Choosing a moderate pH is especially advantageous for samples comprising mRNA, because pH is more important that temperature for denaturing mRNA structure.

The pH used for the separation can be selected to optimize the separation of the nucleic acid in the sample.

The column temperature of the anion-exchange column used in the method of separation according to the present invention is greater than 30° C. In other words, the method of separation according to the present invention is carried out at elevated (above room temperature) column temperatures. In preferred aspects of the present invention, the column temperature is at least 32° C., preferably at least 40° C. Preferably, the column temperature of the anion-exchange column used in the method of separation of the invention is no greater than 60° C.

Alternatively, when performing ion-pair mediated salt gradient methods of the invention as defined above the column temperature of the anion-exchange column used in the method of separation of the invention is no greater than 50° C. and preferably no greater than 45° C.

The column temperature used for the separation can be selected to optimize the separation of the nucleic acid in the sample. For example, the column temperature may be optimized to achieve the highest resolution separation.

In preferred aspects of the invention, a detector can be used to detect components of the eluted sample. For example, a non-destructive detector such as a UV/Vis (ultraviolet/visible) wavelength light detector can be used to detect components of the eluted sample and produce a chromatogram.

In some aspects of the invention, the anion-exchange separation may be followed by one or more further analytical techniques, such as: circular dichroism; mass spectrometry (MS); DSC; qPCR; next generation sequencing (NGS); Sanger sequencing; and capillary electrophoresis (CE); fragment generation via nucleases and subsequent analysis of the fragments by CE or LC-MS. The anion-exchange separation may alternatively or additionally be part of a 2D-LC setup with a desalting step in the second dimension.

### Method for Determining One or More Calorimetric Properties

The present invention provides a method for determining one or more calorimetric properties of a nucleic acid, e.g., a temperature at which self-structure is lost, a melting point, a temperature at which there is a change in secondary structure, etc. Said method comprises carrying out anion-exchange separation on a sample comprising a nucleic acid. The sample is loaded onto an anion-exchange column as described above. For example, the sample may be injected onto the column through an injection valve. Any suitable anion-exchange column may be selected for carrying out the method for determining a calorimetric property according to the invention.

Once the sample comprising a nucleic acid has been loaded onto the anion-exchange column, the sample is eluted from the column at a first column temperature to produce a chromatogram. For example, the sample may be eluted from the column using a salt gradient. A chromatogram may be produced using a UV/Vis wavelength light detector.

The same anion-exchange separation is then repeated at a number of additional column temperatures. Preferably, the anion-exchange separation is repeated at at least three additional column temperatures. At each column temperature, the sample comprising the nucleic used should be identical, i.e., same amount of sample and same concentration of nucleic acid.

After repeating the anion-exchange separation at a number of additional column temperatures, the chromatograms obtained at the different column temperatures are compared to determine one or more calorimetric properties of the nucleic acid present in the sample.

In some aspects of the invention, the comparing may comprise comparing the peak area of corresponding peaks in the chromatograms obtained at the different column temperatures. In other words, the comparing may comprise comparing the peak area of a peak produced by the nucleic acid at the first column temperature to the peak area of corresponding peaks for the additional column temperatures.

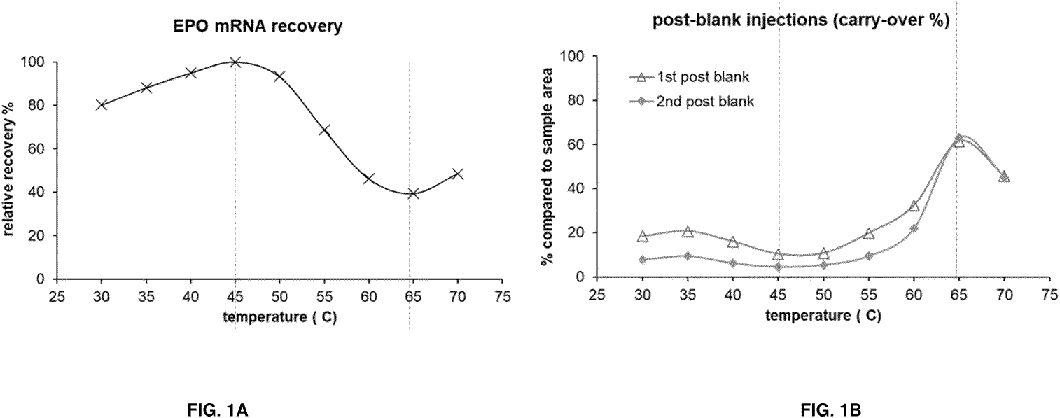

FIG. 1A shows an illustrative plot of relative recovery % against temperature for the anion-exchange separation of a sample comprising EPO mRNA. EPO mRNA encodes the human erythropoietin (EPO) protein.

Anion-exchange chromatography was carried out on a sample comprising EPO mRNA at a range of column temperatures between 30° C. and 70° C. (i.e., 30, 35, 40, 45, 50, 55, 60, 65 and 70° C.) to produce the illustrative plot of FIG. 1A. At each column temperature, a chromatogram was obtained and the peak area of a peak produced by EPO mRNA calculated: the peak area being calculated for the corresponding peak in each chromatogram. The calculated peak areas were then normalised against the largest peak area calculated, to determine a relative recovery % for each column temperature, the relative recovery % for the column temperature at which the calculated peak area was the largest being 100 %. The relative recovery % was then plotted against column temperature.

As shown in FIG. 1A, for EPO mRNA, a peak is seen at approximately 45° C. and a trough at approximately 65° C. in a plot of relative recovery % against column temperature. The peak and trough indicate that EPO mRNA has a calorimetric property, such as a melting temperature, at 45° C. and another calorimetric property at 65° C.

More accurate temperature values could be found by repeating the anion-exchange separation for a number of column temperature values around 45° C. and a number of column temperature values around 65° C.

In some aspects of the invention, the comparing may comprise determining the carry-over % at different column temperatures. In other words, the comparing may comprise comparing the carry-over % for the nucleic acid at the first column temperature to carry-over % for the nucleic acid at the additional column temperatures.

Carry-over is sample that remains on the column after the anion-exchange separation has been completed. Carry-over can be detected by performing blank runs (i.e., elution of the column with no sample injected) after completing the anion-exchange separation of the sample. FIG. 1B shows results obtained by carrying out a first and a second blank run at each column temperature. Any sample peaks which appear in the chromatogram obtained from a blank run must be the result of carry-over.

FIG. 1B shows an illustrative plot of carry-over % against temperature for a sample comprising EPO mRNA. To produce the illustrative plot of FIG. 1B, at each column temperature, the peak area of a peak produced by EPO mRNA in a blank run was compared to the peak area of the corresponding peak in the chromatogram obtained for the anion-exchange separation of the sample comprising EPO mRNA. Carry-over % compared to sample area could then be determined. For example, if the peak area is calculated to be 10 in the blank run and 50 for the sample separation at a particular column temperature, the carry-over % compared to sample area for that column temperature will be 20 % (i.e. (10/50)*100). Carry-over % compared to sample area was then plotted against column temperature.

In the plot of FIG. 1B, a peak is seen at approximately 65° C. and a trough at approximately 45° C. This indicates that EPO mRNA has a calorimetric property, such as a melting temperature, at 45° C. and another calorimetric property at 65° C. In other words, the results from plotting carry-over % against column temperature are consistent with the results obtained by plotting relative recovery % against column temperature.

More accurate temperature values could be found by repeating the anion-exchange separation and determining the carry-over % at a number of column temperature values around 45° C. and a number of column temperature values around 65° C.

Some advantages of the method of the invention for determining one or more calorimetric properties over traditional DSC include that the present method: allows sample fraction collection at the same time as determining one or more calorimetric properties; can be used in combination with orthogonal analytical techniques to find out further information about the nucleic acid in the sample; and typically enables faster determination of one or more calorimetric properties than DSC.

When used in the description and claims, the terms “comprises” and “comprising”, and variations thereof, mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Although certain example embodiments of the invention have been described herein, the scope of the appended claims is not intended to be limited solely to these embodiments. The claims are to be construed literally, purposively, and/or to encompass equivalents.

### Example 1 - An Anion-Exchange Method for Separating a Sample Comprising mRNA Using an NaCl Gradient

In this study, we observed the anion-exchange separation of both Cas9 mRNA and EPO mRNA, using a sodium chloride gradient, at a number of temperatures between 30° C. and 60° C. Cas9 mRNA encodes the CRISPR-associated protein 9 (Cas9).

The anion-exchange column selected for carrying out the separations was a Protein-Pak Hi-Res Q, 100 × 4,6 mmm, 5 µm column (Waters Corporation, Milford, MA, USA). The column was used with a flow rate of 0.6 ml/min. Mobile phase A used in the separations comprised 25 mM Tris buffering agent and had a pH of 7.6. Mobile phase B used in the separation comprised 25 mM Tris buffering agent and 2 M NaCl, and had a pH of 7.6.

For each separation, 5 µl of a sample comprising 20 µg/ml Cas9 or EPO mRNA was injected onto the anion-exchange column. The sample was eluted from the anion-exchange column by gradually increasing the concentration of NaCl on the column. This was achieved by increasing the percentage of mobile phase B from 20 to 70 % over the course of 12 minutes (i.e. ΔB = 4.17 %/min). The eluted sample was detected by a 260 nm UV detector.

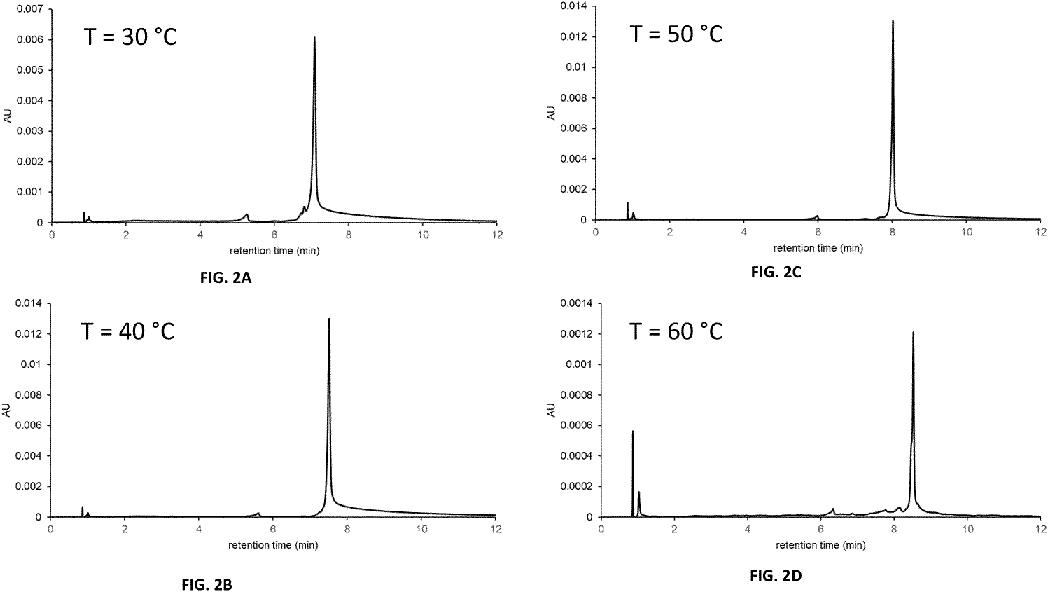

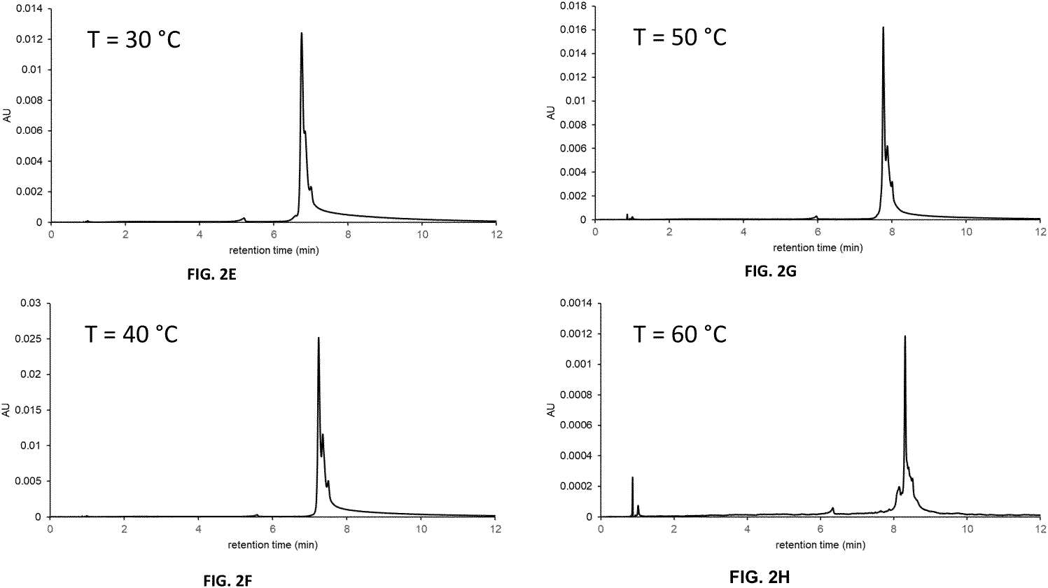

The anion-exchange separation was carried out for a sample comprising Cas9 mRNA at the following column temperatures: 30° C.; 40° C.; 50° C.; and 60° C. The chromatograms obtained from the separations at the various temperatures are shown in FIGS. 2A, 2B, 2C, and 2D.

The anion-exchange separation was also carried out for a sample comprising EPO mRNA at the following column temperatures: 30° C.; 40° C.; 50° C.; and 60° C. The chromatograms obtained from the separations at the various temperatures are shown in FIGS. 2E, 2F, 2G and 2H.

As can be seen from FIGS. 2A-2D and FIGS. 2E-2H, for both Cas9 mRNA and EPO mRNA, the highest resolution separation was achieved using a column temperature of 60° C. Peak tailing is also minimized for both Cas9 mRNA and EPO mRNA when using a column temperature of 60° C.

### Example 2- An Anion-Exchange Method for Separating a Sample Comprising mRNA Using a TMAC Gradient

In this study, we observed the anion-exchange separation of both Cas9 mRNA and EPO mRNA using a tetramethylammonium chloride (TMAC) gradient at a number of temperatures between 30° C. and 60° C.

The anion-exchange column selected for carrying out the separations was again a Protein-Pak Hi-Res Q, 100 × 4,6 mmm, 5 µm column (Waters Corporation, Milford, MA, USA). The column was again used with a flow rate of 0.6 ml/min. Mobile phase A used in the separations comprised 25 mM Tris buffering agent and had a pH of 7.6. Mobile phase B used in the separation comprised 25 mM Tris buffering agent and 3 M TMAC, and had a pH of 7.6.

For each separation, 5 µl of sample comprising 20 µg/ml Cas9 or EPO mRNA was injected onto the anion-exchange column. The sample was eluted from the anion-exchange column by gradually increasing the concentration of TMAC on the column. This was achieved by increasing the percentage of mobile phase B from 60 to 100 % over the course of 10 minutes (i.e. ΔB = 4 %/min). The eluted sample was detected by a 260 nm UV detector.

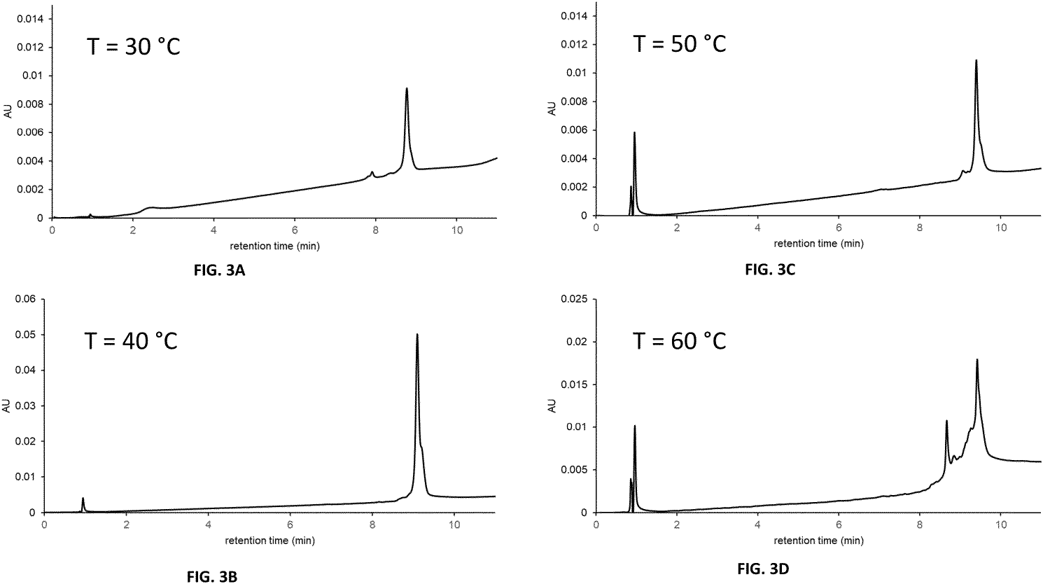

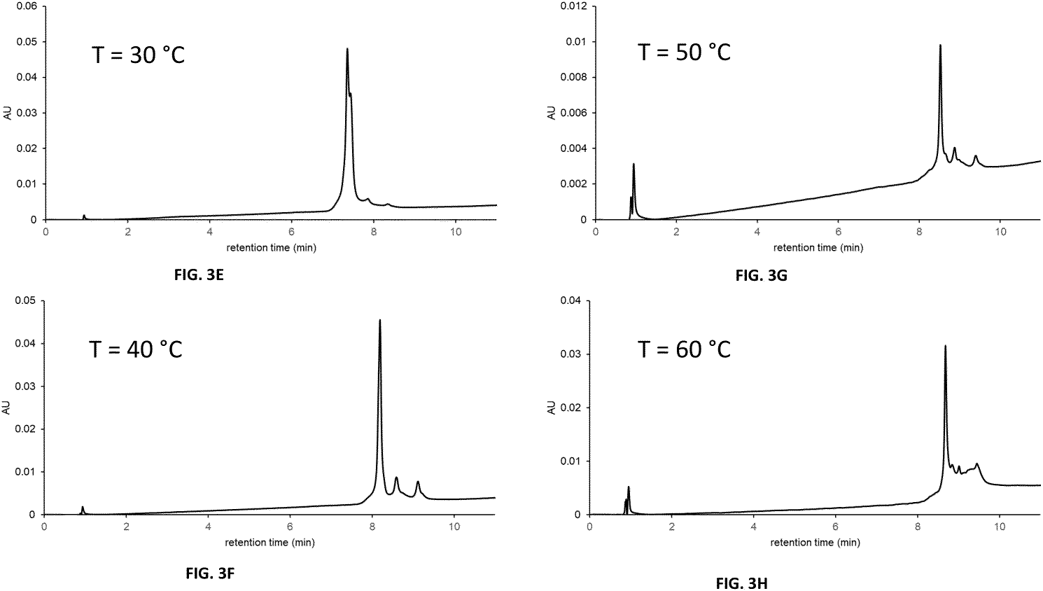

The anion-exchange separation was carried out for a sample comprising Cas9 mRNA at the following column temperatures: 30° C.; 40° C.; 50° C.; and 60° C. The chromatograms obtained from the separations at the various temperatures are shown in FIGS. 3A, 3B, 3C, and 3D.

The anion-exchange separation was also carried out for a sample comprising EPO mRNA at the following column temperatures: 30° C.; 40° C.; 50° C.; and 60° C. The chromatograms obtained from the separations at the various temperatures are shown in FIGS. 3E, 3F, 3G, and 3H.

As can be seen from FIGS. 3A-3D and FIGS. 3E-3H, for both Cas9 mRNA and EPO mRNA, the highest resolution separation was achieved using a column temperature of 40° C. In other words, a high resolution was achieved at a lower column temperature when using a TMAC salt gradient to elute the sample, than when using a NaCl salt gradient (c.f. FIGS. 2A-2D and FIGS. 2E-2H to FIGS. 3A-3D and FIGS. 3E-3H).

Additionally, as can be seen most clearly in the chromatogram obtained for a column temperature of 40° C. but can also be seen in the chromatogram obtained for a column temperature of 50° C., baseline resolution of two acidic variants of EPO mRNA was observed using a TMAC salt gradient to elute the sample (see the two peaks eluting after the main peak in the 40° C. and 50° C. chromatograms of FIGS. 3E-3H). Baseline resolution of these two acidic variants was not achieved using a NaCl salt gradient at any temperature (see FIGS. 2E-2H, where two distinct peaks eluting after the main peak are not observed in any of the chromatograms). Accordingly, the use of a TMAC salt gradient achieves a higher resolution than the use of a NaCl salt gradient.

This example shows that use of a salt comprising a mild ion-paring cation, such as tetramethylammonium chloride, to produce a salt gradient for elution of a sample comprising a nucleic acid, such as mRNA, in anion-exchange chromatography, allows a very high resolution to be achieved. Furthermore, in the present example, a high resolution is achieved in conditions which are stabilising for the nucleic acid in the sample (i.e., 40° C. and moderate pH).

A higher, less stabilising temperature (60° C.) is required to achieve a high resolution separation using a NaCl salt gradient. Furthermore, the separation achieved using NaCl and a column temperature of 60° C. is not as high resolution: it is not possible to resolve peaks for the two acidic variants of EPO mRNA using a NaCl salt gradient.

A very high pH was required in Kanavarioti (e.g., pH 12) to achieve a high resolution of a sample comprising RNA at a low temperature. Here, a high resolution has been achieved at only 40° C. and using a neutral pH (e.g., pH 7.6).

### Example 3 - An Anion-Exchange Method for Separating a Sample Comprising mRNA Using an NaCl Gradient in the Presence of TMAC (“Ion Pair Mediated Salt Gradient”)

In this study, we observed the anion-exchange separation of both Cas9 mRNA and EPO mRNA, using a sodium chloride gradient, at a number of temperatures between 30° C. and 70° C. Additionally TMAC was provided as an additive in the mobile phase at a static concentration of 1 M.

The anion-exchange column selected for carrying out the separations was a custom-packed 50 × 2.1 mm, 5 µm Protein-Pak Hi Res Q strong anion exchange column (Waters Corporation, Milford, MA, USA). The column was used with a flow rate of 0.2 ml/min. Mobile phase A used in the separations comprised 25 mM Tris buffering agent with pH adjusted to 7.5 to 8.0 through the addition of 1 N HCl or 1 N NaOH solutions, respectively. Mobile phase B used in the separation comprised 25 mM Tris buffering agent and 2 M NaCl, and had a pH of 7.6. Mobile phase C used in the separations comprised 25 mM Tris buffering agent and 3 M TMAC.

For each separation, 1 µl of sample comprising 50 µg/ml Cas9 or EPO mRNA was injected onto the anion-exchange column. The sample was eluted from the anion-exchange column by gradually increasing the concentration of NaCl on the column. This was achieved by increasing the percentage of mobile phase B from 14 to 44 % over the course of 4.75 minutes (i.e. ΔB = 6.32 %/min). The mobile phase also comprised a static concentration of TMAC of 1.0 M, from mobile phase C. The eluted sample was detected by a 260 nm UV detector.

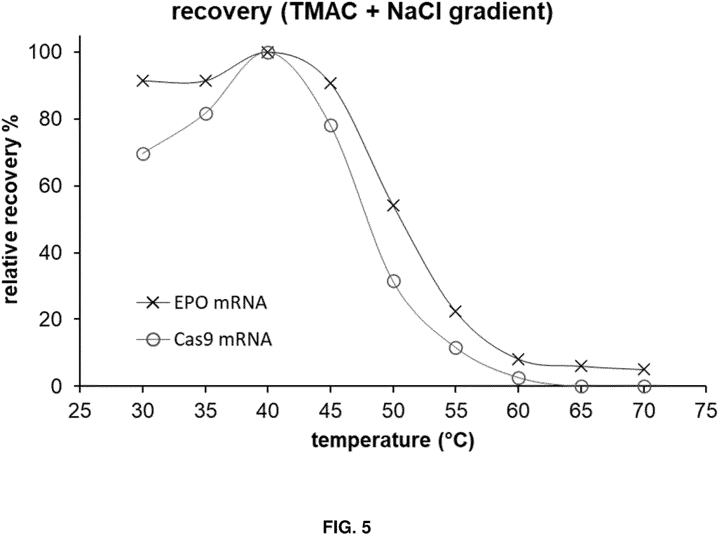

The anion-exchange separation was carried out for a sample comprising Cas9 mRNA and for a sample comprising EPO mRNA at the following column temperatures: 30° C.; 40° C.; 50° C., 60° C. and 70° C. FIG. 5 shows their recovery as a function of temperature and it is clear that temperatures between 30 and 45° C. are advantageous. At high temperatures (T ≥ 55° C.), the mRNAs could not be readily eluted from the column

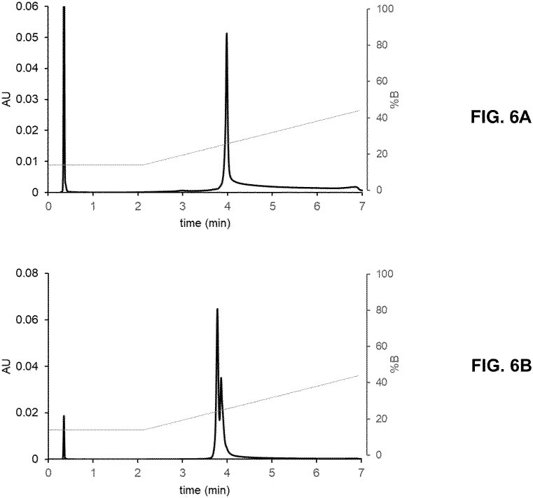

FIGS. 6A-6B show chromatograms at 40° C. for Cas9 mRNA and EPO mRNA. As clearly shown this elution mode resulted in very well-resolved peaks which can be attributed to the tuning of the effective number of charges directly involved in solute to stationary phase interactions.

Additionally, Table 1 below summarizes the retention model parameters observed for the ion-pair mediated salt gradient experiment described above (static concentration of TMAC = 1.0 M) compared with a concentration of 0.3 M TMAC. When using a constant ion-pairing agent concentration (TMAC at relatively low - 0.3 M—- and at relatively high - 1 M - concentration as mobile phase additive) and running a NaCl gradient (ion-pair mediated salt gradient mode) then significantly lower amounts of NaCl could be used to effectively elute the compounds. At a 0.3 M TMAC additive concentration, 0.85 M NaCl produced elution conditions. At a 1.0 M TMAC concentration, only 0.58 - 0.59 M NaCl was needed to elute the mRNA samples. Even in this mixed mode separation on-off like behaviour was observed. In other words, the solute retention is sensitive to the mobile phase composition and the described method allows the adjustment of selectivity by modifying the mobile phase

TABLE 1

| | EPO mRNA | Cas9 mRNA |
|---|---|---|
| Molecular Size (nt) | 858 | 4521 |
| 0.3 M TMAC + NaCl gradient | | |
| S | 54.4 | 58.2 |
| c* (%B eluent) | 42.7%B | 42.3%B |
| salt required (M) to elute (k=1) | 0.85 | 0.85 |
| 1.0 M TMAC + NaCl gradient | | |
| S | 86.0 | 95.6 |
| c* (%B eluent) | 28.9%B | 28.8%B |
| salt required (M) to elute (k=1) | 0.58 | 0.58 |

It is also considered that a high resolution separation, wherein the peaks for the two acid variants of EPO mRNA are resolved, may result from using a dual gradient, i.e. a NaCl salt gradient and a salt gradient of a salt comprising a mild ion-pairing cation. This has the potential to further adjust selectivity and to introduce more pronounced gradient band compression effects. In both of these scenarios (static concentration of mild ion-pairing cation and dual gradient), the mild ion-pairing cation is still able to exert an ion-pairing effect on the nucleic acid in the sample.

### Example 4 - An Anion-Exchange Method for Separating Samples Comprising ssRNA and sgRNA Using an TMAC Gradient

In this study, we observed the anion-exchange separation of samples comprising: a low range ssRNA ladder (50 to 1000 nucleotides); HPRT sgRNA (100 nucleotides); GUAC ssRNA (150 nucleotides); Rosa26 sgRNA (100 nucleotides); and Scramble #2 sgRNA (100 nucleotides) using a tetramethylammonium chloride (TMAC) gradient and a column temperature of 60° C. The low range ssRNA ladder sample was purchased from New England Biolabs; both the HPRT sgRNA and GUAC ssRNA samples were purchased from Integrated DNA Technologies (IDT); and both the Rosa26 sgRNA and Scramble #2 sgRNA samples were purchased from Synthego.

HPRT sgRNA, Rosa26 sgRNA, and Scramble #2 sgRNA are single guide RNAs (sgRNAs) comprising a crRNA and a tracrRNA sequence, wherein the crRNA and tracrRNA sequences direct the Cas9 protein to a specific DNA site. GUAC ssRNA is a single-stranded RNA (ssRNA) comprising repeats of the GUAC base sequence. The low range ssRNA ladder comprises a number of ssRNA chains of differing length between 50 to 1000 nucleotides in length.

The anion-exchange column selected for carrying out the separations was a Protein-Pak Hi-Res Q, 100 × 4,6 mmm, 5 µm column (Waters Corporation, Milford, MA, USA). The column was used with a flow rate of 0.4 ml/min. Mobile phase A used in the separations comprised 100 mM Tris-HCl; mobile phase B used in the separations comprised 100 mM Tris; mobile phase C used in the separations comprised 3 M TMAC; and mobile phase D used in the separations comprised water. The mobile phases were buffered at pH 9 using 20 mM Tris buffering agent.

For each separation, 1-10 µl of sample was injected onto the anion-exchange column. The sample was eluted from the anion-exchange column using a TMAC gradient.

Table 2 below shows how the % of each of the four mobile phases was varied throughout the separations to create a TMAC gradient. The initial concentration of TMAC is set to 0 mM, to ensure that the sample strongly binds to the anion-exchange column. After 5 minutes, the percentage of mobile phase C comprising TMAC is increased to 46.7 %. The column is allowed to equilibrate at 46.7 % C for 4 minutes, before the percentage of mobile phase C is increased linearly to 70% over the course of 20 minutes (i.e. ΔC = 1.17 %/min). The percentage of mobile phase C is then increased further to 80 % over the course of 2 minutes.

TABLE 2

Gradient table

| Time (min) | %A | %B | %C | %D |
|---|---|---|---|---|
| 0 | 3.3 | 16.7 | 0.0 | 80.0 |
| 5 | 3.3 | 16.7 | 46.7 | 33.3 |
| 9 | 3.3 | 16.7 | 46.7 | 33.3 |
| 29 | 3.3 | 16.7 | 70.0 | 10.0 |
| 31 | 3.3 | 16.7 | 80.0 | 0.0 |
| 33 | 3.3 | 16.7 | 80.0 | 0.0 |
| 33.1 | 3.3 | 16.7 | 0.0 | 80.0 |
| 45 | 3.3 | 16.7 | 0.0 | 80.0 |

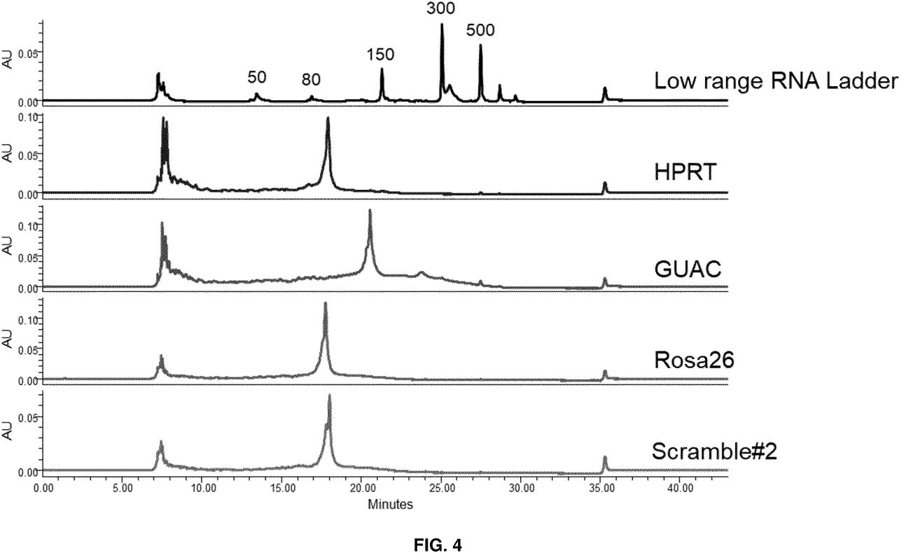

The eluted sample was detected by a 260 nm UV detector. The chromatograms obtained from the separations are shown in FIG. 4.

As can be seen from FIG. 4, a high resolution separation with minimal peak tailing was achieved for each of the samples at the column temperature of 60° C.

This example shows that the anion-exchange separation of the present invention can be used to separate relatively short nucleic acids. In example 2, a high resolution separation was achieved for Cas9 mRNA (4521 nucleotides) and EPO mRNA (858 nucleotides). In the present example, a high resolution separation has been achieved for nucleic acids as short as 100 nucleotides in length.

The anion-exchange separation was repeated for each of the samples using a pH 7.4 Tris buffering agent. High resolution separations were also achieved at this lower pH. Accordingly, it is possible to achieve a high resolution separation for each of the samples at near neutral pH.

Similar to in example 2, the resolution of the separation may be further optimised by testing different column temperatures above 30° C. It is possible that a higher resolution could be obtained for each of the ssRNA and sgRNA samples at a column temperature below 60° C.

It is also possible that a high resolution separation could be achieved for each of the ssRNA and sgRNA samples using a non-ion-pairing salt, such as NaCl or NaBr, to perform the salt gradient in the presence of a static concentration of mild ion-pairing cation, such as tetramethylammonium. Similarly, it is possible that a high resolution separation could be achieved for each of the ssRNA and sgRNA samples using dual gradient: a salt gradient of a non-ion-pairing salt and a salt gradient of a salt comprising a mild ion-pairing cation.

1. A method for separating a sample comprising a nucleic acid, the method comprising:
(a) loading a sample comprising a nucleic acid onto an anion-exchange column; and
(b) eluting the sample from the anion-exchange column using a salt gradient,
wherein a mobile phase used in the eluting step comprises a mild ion-pairing cation, and wherein a column temperature of the anion-exchange column is greater than 30° C.

2. The method of claim 1, wherein the salt gradient is produced by a salt comprising the mild ion-pairing cation.

3. The method of claim 1, wherein the salt gradient is produced by a non-ion-pairing salt, and wherein a concentration of the mild ion-pairing cation remains constant throughout the eluting step.

4. The method of claim 3, wherein the concentration of the mild ion-pairing cation is 10 to 2,000 mM.

5. The method of claim 3, wherein the column temperature of the anion-exchange column does not exceed 50° C.

6. The method of claim 1, wherein the salt gradient is produced by: a salt comprising the mild ion-pairing cation; and a non-ion-pairing salt, preferably a chloride or bromide containing salt.

7. The method of claim 1, wherein the nucleic acid is RNA.

8. (canceled)

9. The method of claim 1, wherein the method uses at least two mobile phases, and wherein the mobile phases are buffered with a basic or a zwitterionic buffering agent.

10. The method of claim 9, wherein the buffering agent is selected from Tris, Bis-tris propane, MES, and HEPES.

11. The method of claim 10, wherein the buffering agent is Tris.

12. The method of claim 1, wherein the mild ion-pairing cation is nitrogenous.

13. The method of claim 1, wherein the mild ion-pairing cation is tetramethylammonium.

14. The method of claim 2, wherein the salt gradient is produced by tetramethylammonium chloride.

15. The method of claim 14, wherein the column temperature of the anion-exchange column is 40° C.

16. The method of claim 1, wherein the method uses at least two mobile phases, and wherein the mobile phases have a pH of from 6.5 to 10.

17. The method of claim 16, wherein the mobile phases have a pH of from 7 to 9.

18. The method of claim 1, wherein the method uses at least two mobile phases, and wherein one or more of the at least two mobile phases comprises at least one additional mild ion-pairing cation.

19. The method of claim 18, wherein the at least one additional mild ion-pairing cation is selected from tetramethylammonium, triethylammonium, diisopropylethylammonium, or combinations thereof.

20. A method for determining one or more calorimetric properties of a nucleic acid, the method comprising:
(a) loading a sample comprising a nucleic acid onto an anion-exchange column;
(b) eluting the sample from the anion-exchange column at a first column temperature to produce a first chromatogram;
(c) repeating steps (a) and (b) for a plurality of additional column temperatures to produce a corresponding plurality of additional chromatograms; and
(d) comparing the chromatograms obtained at different column temperatures to determine one or more calorimetric properties of the nucleic acid.

21-26. (canceled)

27. The method of claim 1, wherein the salt gradient is produced by a non-ion-pairing salt selected from a chloride containing salt and bromide containing salt.
